(19) United States
(12) Patent Application Publication
(10) Pub. No.: US 2018/0174683 A1
(43) Pub. Date: Jun. 21, 2018

(54) DEVICE, METHOD AND COMPUTER PROGRAM FOR CONFIGURING A MEDICAL DEVICE, MEDICAL DEVICE, METHOD AND COMPUTER PROGRAM FOR A MEDICAL DEVICE

(71) Applicant: Drägerwerk AG & Co. KGaA, Lubeck (DE)

(72) Inventors: Frank FRANZ, Stockelsdorf (DE); Stefan SCHLICHTING, Lubeck (DE); Jasper DIESEL, Lubeck (DE)

(21) Appl. No.: 15/845,080

(22) Filed: Dec. 18, 2017

(30) Foreign Application Priority Data
Dec. 20, 2016 (DE) 10 2016 015 119.6

(51) Int. Cl. G16H 40/63; A61B 5/00; G16H 40/67

(52) U.S. Cl. CPC G16H 40/63; G16H 30/20; G16H 40/67; A61B 5/0015

(57) ABSTRACT

A device (10), a method and a computer program for configuring a medical device (20), a medical device (20), a method and a computer program for a medical device are provided. The device (10) includes at least one interface (12) for communication with the at least one medical device (20) and for receiving optical image data of the medical device and of an area surrounding the medical device. The device (10) further comprises a computer (14) for controlling the at least one interface (12) and for determining whether a user of the medical device (20) is located in the area surrounding the medical device. The computer (14) is further configured to communicate with the medical device when the user is located in the area surrounding the medical device (20). The computer is configured to receive addressing information about the at least one medical device via the at least one interface.

### CROSS REFERENCE TO RELATED APPLICATIONS

This application claims the benefit of priority under 35 U.S.C. § 119 of German Application 10 2016 015 119.6, filed Dec. 20, 2016, the entire contents of which are incorporated herein by reference.

### FIELD OF THE INVENTION

Exemplary embodiments pertain to a device, to a method and to a computer program for configuring a medical device, to a medical device, to a method and to a computer program for a medical device, but especially but not exclusively, to a concept for the automated configuration of medical devices based on optical image data.

### BACKGROUND OF THE INVENTION

In health care, nurses and health care staff process many different types of information. This information is provided, for example, by user interfaces of different devices. Examples of such data are physiological parameters, such as blood pressure, heart rate, oxygen saturation, etc., which are provided by corresponding monitoring devices with monitors, displays or control lights and control displays. The information provided should be readily accessible and able to be accessed or interpreted at any time, because the health care staff shall be informed rapidly and reliably in critical situations in order to be able to take the correct actions, and it shall be enabled to have the ability to develop a feeling for the health status of the patient.

User interfaces (UI) can communicate or pass on information to health care staff, e.g., in the form of graphic representations, displays of parameters, warning signals (optical/acoustic), etc. Patient monitoring devices or monitors are typical examples. These devices may make possible a continuous monitoring of a plurality of parameters. Examples are heart rate, respiration rate, blood pressure, oxygen saturation, body temperature, etc. Such devices are often used in intensive care units, in operating rooms, in hospital rooms or for sedated patients.

Other devices with displays, display units or other user interfaces are, for example, ventilators, anesthesia workplaces, incubators, dialysis machines, etc. All these devices have certain parameters, which are set and monitored by the health care staff. Some of these devices also have devices to draw the attention of the health care staff, for example, alarm lights or alarm sounds. Physical interaction, e.g., by means of pushbuttons and slider controls, are widely used as well. The medical devices are usually preset or configured in this case by the health care staff, so that the data relevant to the particular case can be read or accessed.

Additional background information can be found in the following documents:
Besl, P. J. (1992), “Method for registration of 3-D shapes,”, in: Robotics-DL tentative (pp. 586-606).
Fischler, M. A. (1981), “Random sample consensus: a paradigm for model fitting with applications to image analysis and automated cartography,” Communications of the ACM, pp. 381-395.
Hartman, F. (2011), “Robot control by gestures,” Master's thesis, University of Lubeck.
Kong, T. & Rosenfeld, A. (1986), “Topological Algorithm for Digital Image Processing,”, Elsevier Science, Inc.
Shapiro, L., & Stockman, G. (2001), “Computer Vision,” Prentice-Hall.
Besl, Paul J.; N. D. McKay (1992), “A Method for Registration of 3-D Shapes,” IEEE Trans. on Pattern Analysis and Machine Intelligence (Los Alamitos, Calif., USA: IEEE Computer Society) 14 (2): 239-256.
Alexandre, Luis A., “3D descriptors for object and category detection: a comparative evaluation,” Workshop on Color-Depth Camera Fusion in Robotics at the IEEE/RSJ International Conference on Intelligent Robots and Systems (IROS), Vilamoura, Portugal, Vol. 1, No. 3, 2012.
Woodford, Oliver J. et al., “Demisting the Hough transform for 3D shape detection and registration,” Internal Journal of Computer Vision, 106.3 (2014): 332-341.
Velizhev, Alexander, Roman Shapovalov, and Konrad Schindler, “Implicit shape models for object detection in 3D point clouds,” International Society of Photogrammetry and Remote Sensing Congress, Vol. 2, 2012.
S. Gupta, R. Girshick, P. Arbelaez, and J. Malik, “Learning rich features from RGB-D images for object detection and segmentation,” In ECCV, 2014.
Gupta, Saurabh, et al., “Aligning 3D models to RGB-D images of cluttered scenes,” Proceedings of the IEEE Conference on Computer Vision and Pattern Detection, 2015.
S. Song and J. Xiao, “Sliding Shapes for 3D Object Detection in Depth Images,” In ECCV, 2014.
Song, Shuran, and Jianxiong Xiao, “Deep Sliding Shapes for Amodal 3D Object Detection in RGB-D Images,” arXiv preprint arXiv:1511.02300 (2015).
Tombari, S., Salti, S., & Di Stefano, L. (2010), “Unique Signatures of Histograms for Local Surface Description,” Proceedings of the 11th European Conference on Computer Vision (ECCV).
Tombari, S., Salti, S., & Di Stefano, L. (2011), “A Combined Texture-Shape Descriptor for Enhanced 3D Feature Matching,” Proceedings of the 18th International Conference on Image Processing (ICIP).
Viola, P., & Jones, M. (2001), “Rapid object detection using a boosted cascade of simple features,” CONFERENCE ON COMPUTER VISION AND PATTERN DETECTION, 2001.
Shotton, J. (2013), “Real-time human pose detection in parts from single depth images,” Communications of the ACM.
Fanelli, G., Weise, T., Gall, J., & Van Gool, L., 2011, “Real time head pose estimation from consumer depth cameras,” Pattern Detection.
Seitz, Steven Maxwell, “Image-based transformation of viewpoint and scene appearance,” Diss. UNIVERSITY OF WISCONSIN (MADISON, 1997).

### SUMMARY OF THE INVENTION

Therefore, there is a need for developing an improved concept for a configuration of a medical device. This need is met by a device, a method and a computer program for configuring a medical device, a medical device, a method and a computer program for a medical device according to the invention. Exemplary embodiments are based on the discovery that medical devices can be provided with communication interfaces that can be used for communication between a configuration device and the medical devices. A configuration device may take advantage of image-processing and image detection devices in order to detect the medical devices in a detection area and to address them via corresponding networks or interfaces when needed.

Exemplary embodiments create a device for configuring at least one medical device. The device comprises at least one interface for communication with at least one medical device and for receiving optical image data of the medical device and of an area surrounding the medical device. The device further comprises a computer for controlling the at least one interface and for determining whether a user of the medical device is located in the area surrounding the medical device. The computer is further configured to communicate with the medical device when the user is located in the area surrounding the medical device. The computer is configured to receive addressing information about the at least one medical device via the at least one interface. Exemplary embodiments can thus make it possible when needed to automatically provide information for a medical device, for example, information about a user, a distance of a user from the medical device, configuration or setting information for the medical device, etc.

In some exemplary embodiments, the addressing information may comprise, for example, one or more elements of the group of information about a type of the medical device, information about a network address of the medical device, information about a specification of the medical device or information about the ability to reach the medical device or the configuration of the medical device.

Moreover, the device may comprise in exemplary embodiments a detection device for detecting the optical image data of the medical device and of the area surrounding the medical device, wherein the detection device has one or more sensors, which is/are configured to detect a three-dimensional point cloud as image data. Exemplary embodiments can thus make possible an automated image data detection.

The computer may be configured to detect the at least one medical device in the image data and to determine the position of the at least one medical device. It may thus be possible to determine a need for communication with the medical device or a need for setting or configuring the medical device. The computer may be configured to set the at least one medical device for operation by the user and/or for outputting information for the user when the user is located in the area surrounding the at least one medical device. For example, the computer may be configured to determine a distance between the user and the at least one medical device from the image data. If a user is located in the vicinity of the medical device, this medical device can then be set in respect to or for the user in some exemplary embodiments.

The computer may be configured in further exemplary embodiments to determine a viewing direction, a field of view and/or a body orientation of the user in order to infer the presence of an operating and/or reading intention of the user from the viewing direction and/or the body orientation, and to set the medical device for the operating or reading intention when this is present. The computer may also be configured to build up a link to the at least one medical device via the at least one interface when a user is located in the area surrounding the at least one medical device. The build-up of the link can then make possible a corresponding communication.

In some exemplary embodiments, the device may further comprise a storage device, which is configured to store data, wherein the computer may be configured to store information about the medical device by means of the storage device. For example, a future configuration or setting may then be facilitated or expedited by the storage. The computer may be configured to store a time stamp with the information about the medical device and thus to make possible an automated documentation. The computer may also be configured to determine the absence of the user in the area surrounding the at least one medical device and to change a setting of the at least one medical device when the absence of the user was determined. For example, a display or an acoustic output may be faded out or switched off in the absence of the user in order not to disturb a possible patient any longer. It would also be possible to generate alarms or warning signals in another variant in order to make them audible outside a hospital room for the health care staff or a user.

The computer may be configured in some exemplary embodiments to receive an identification from the medical device. The identification can make it possible to identify or detect the medical device. The identification may correspond, for example, to an optical signal, which is detectable in the image data, and/or to an acoustic signal. The computer may be configured to locate and/or to identify the medical device on the basis of the identification and the image data. The computer may be configured to send a trigger signal for sending an identification to the at least one medical device via the at least one interface. A detection process or a registration process between the device and the medical device can be simplified hereby.

In some other exemplary embodiments, the computer may be configured to receive via the at least one interface a registration signal of the at least one medical device, which signal indicates that the at least one medical device would like to register itself. The computer may further be configured to receive an identification of the at least one medical device subsequent to the registration signal. The computer may be configured to detect the registration signal in the image data or to receive the identification from the at least one medical device via the at least one interface subsequent to the registration signal. A registration or detection process can also be automated to this extent.

The image data may comprise infrared image data in exemplary embodiments. The processes being described here may then be carried out independently from the light conditions, for example, during daytime and at night. The at least one interface may further be configured to receive audio data, and the computer may be configured to analyze the audio data with respect to an audio identification of the at least one medical device. Exemplary embodiments can thus also make possible a detection via an audio signal.

In other exemplary embodiments, the computer may be configured to detect a touching and/or interaction of the at least one medical device by or with the user on the basis of the image data and to communicate with the at least one medical device based on a detected touching/interaction by the user. In case of touching/interaction of the medical device by a user, an automated communication or setting of the medical device may then take place. The computer may also be configured to determine on the basis of the image data that the user is located in the area surrounding a plurality of medical devices and to use another medical device based on a detected interaction of the user with a medical device. For example, the interaction of the user with a medical device may also have effects on another medical device, for example, in the interaction between a display and an input device. The device may thus also be configured to set a plurality of medical devices for one or more users.

Exemplary embodiments further provide a medical device that is configured to receive information about a user via a network or an interface, wherein the information about the user indicates whether the user is located in an area surrounding the medical device, wherein the medical device is configured to set display information for a data output based on the information about the user. The medical device may be configured to receive a trigger signal via the network and to send an identification as a response to the trigger signal. The medical device may further be configured to send a registration signal for the medical device in order to register itself with a computer. The medical device may further be configured to send an identification of the at least one medical device subsequent to the registration signal. The identification may be, for example, an optical and/or acoustic signal.

Exemplary embodiments further create a method for configuring at least one medical device. The method comprises the reception of optical image data of the medical device and of an area surrounding the medical device. The method further comprises the reception of addressing information about the at least one medical device and a determination of whether a user of the medical device is located in the area surrounding the medical device. The method comprises a communication with the at least one medical device when the user is located in the area surrounding the medical device.

Exemplary embodiments also create a method for a medical device. The method comprises the reception of information about a user, e.g., via a network and/or an interface, wherein the information about the user shows whether the user is located in an area surrounding the medical device. The method further comprises the setting of display information for a data output based on the information about the user.

Moreover, exemplary embodiments create a program with a program code for executing at least one of the methods described here when the program code is executed on a computer, a processor or a programmable hardware component.

Further advantageous embodiments will be described in more detail below on the basis of the exemplary embodiments shown in the drawings, even though the advantageous embodiments are not generally limited to these exemplary embodiments.

The various features of novelty which characterize the invention are pointed out with particularity in the claims annexed to and forming a part of this disclosure. For a better understanding of the invention, its operating advantages and specific objects attained by its uses, reference is made to the accompanying drawings and descriptive matter in which preferred embodiments of the invention are illustrated.

### BRIEF DESCRIPTION OF THE DRAWINGS

In the drawings:

FIG. 1 is a schematic view showing an exemplary embodiment of a device for configuring a medical device and an exemplary embodiment of a medical device;

FIG. 2 is an overview diagram for determining three-dimensional image data in some exemplary embodiments;

FIG. 3 is a flow chart of a method in an exemplary embodiment;

FIG. 4 is schematic view showing an exemplary embodiment in a hospital room;

FIG. 5 is a flow chart for registering, detecting or linking medical devices via a network with the use of optical or acoustic identifications in an exemplary embodiment;

FIG. 6 is a flow chart for registering, detecting or linking medical devices via a network with detection of manipulations with an object in an exemplary embodiment;

FIG. 7 is a flow chart for determining distances between a health care staff member and a medical device;

FIG. 8 is a flow chart for determining a viewing direction or a field of view in an exemplary embodiment;

FIG. 9 is a flow chart for adapting a user interface in an exemplary embodiment;

FIG. 10 is a flow chart for avoiding frequent adaptations of a user interface;

FIG. 11 is a flow chart for adapting a user interface of a medical device A based on a change in a parameter at a medical device B in an exemplary embodiment;

FIG. 12 is a block diagram of a flow chart of an exemplary embodiment of a method for configuring a medical device; and

FIG. 13 is a block diagram of a flow chart of an exemplary embodiment of a method for a medical device.

### DESCRIPTION OF THE PREFERRED EMBODIMENTS

Referring to the drawings, various exemplary embodiments will now be described in more detail with reference to the attached drawings, in which some exemplary embodiments are shown.

In the following description of the attached figures, which show only some examples of exemplary embodiments, identical reference numbers may designate identical or comparable components. Further, summary reference numbers may be used for components and objects that occur multiple times in an exemplary embodiment or in a drawing, but are described together in respect to one or more features. Components or objects that are described with the same or summary reference numbers, may have identical, but optionally also different configurations in respect to individual, a plurality of or all features, for example, their dimensioning, unless something different appears explicitly or implicitly from the description. Optional components are shown with broken lines or arrows in the figures.

Even though exemplary embodiments may be modified and varied in different ways, exemplary embodiments are shown as examples in the figures and are described here in detail. It shall, however, be made clear that exemplary embodiments are not intended to be limited to the respective disclosed forms, but exemplary embodiments shall rather cover all functional and/or structural modifications, equivalents and alternatives, which are within the scope of the present invention. Identical reference numbers designate identical or similar elements in the entire description of the figures.

It should be noted that an element, which is described as being “connected” or “coupled” to another element, may be connected or coupled to the other element directly or that intermediate elements may be present. If, by contrast, an element is described as being “directly connected” or “directly coupled” to another element, no intermediate elements are present. Other terms, which are used to describe the relationship between elements, should be interpreted similarly (e.g., “between” versus “directly in between,” “adjoining” vs. “adjoining directly,” etc.).

The terminology that is used here is used only to describe certain exemplary embodiments and shall not limit the exemplary embodiments. As used here, the singular forms “a, an” and “the” shall also include the plural forms unless the context unambiguously indicates something else. Further, it shall be made clear that terms such as “contain,” “containing,” “has,” “comprises,” “comprising” and/or “having,” as used here, indicate the presence of said features, integers, steps, work processes, elements and/or components, but they do not exclude the presence or the addition of a feature of or a plurality of features, integers, steps, work processes, elements, components and/or groups thereof.

Unless defined otherwise, all the terms used here (including technical and scientific terms) have the same meaning that a person skilled in the art in the field to which the exemplary embodiments belong attaches to them. It shall further be made clear that terms, e.g., those that are defined in generally used dictionaries, are to be interpreted as if they had the meaning that is consistent with their meaning in the context of the pertinent technology, and not to be interpreted in an idealized or excessively formal sense, unless this is expressly defined here otherwise.

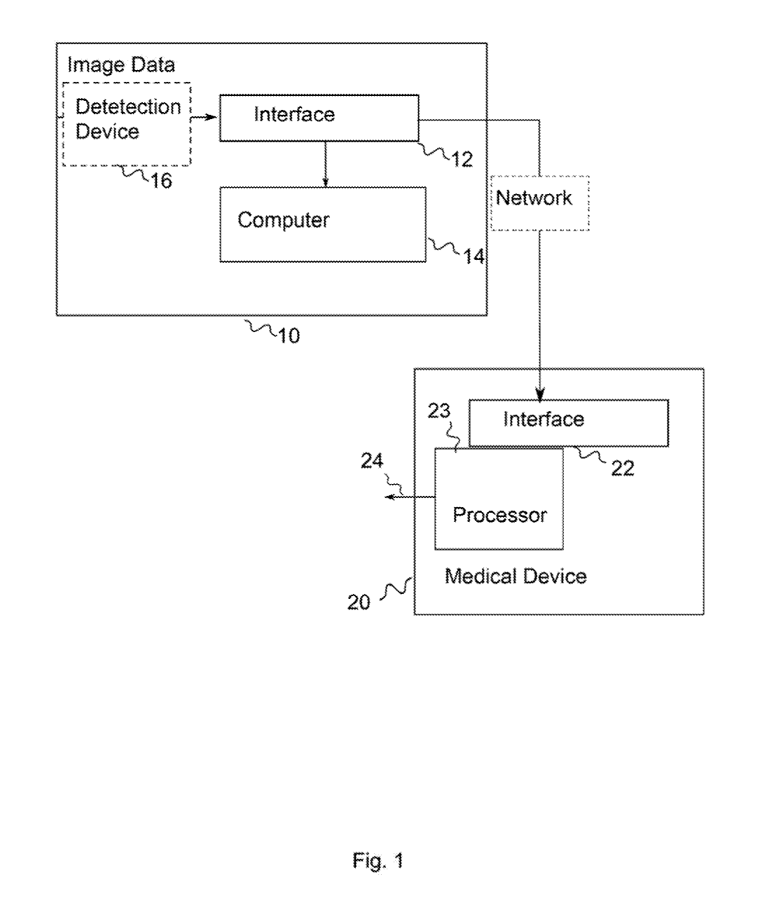

FIG. 1 shows an exemplary embodiment of a device 10 for configuring a medical device 20 and an exemplary embodiment of a medical device 20. The device 10 is adapted to configure at least one medical device 20. The device 10 comprises at least one interface 12 for communication with at least one medical device 20 and for receiving optical image data of the medical device 20 and of an area surrounding the medical device 20. The device 10 further comprises a computer 14 for controlling the at least one interface 12 and for determining whether a user of the medical device 20 is located in the area surrounding the medical device 20. The computer 14 is further configured to communicate with the medical device 20 when the user is located in the area surrounding the medical device 20. The computer 14 is also configured to receive addressing information about the at least one medical device 20 via the at least one interface 12.

Optional components are shown by broken lines in the figures. The interface 12 is coupled to the computer 14. For example, information about the configuration/location of the room to be observed, optionally of a patient positioning device (e.g., angle, intersection angle, information derived therefrom, etc.) and/or information about the addressing of the medical device 20, may be received via the interface 12. A plurality of interfaces 12 or separate interfaces 12 may also be present in some exemplary embodiments in order to receive the image data, on the one hand, to receive the addressing information and in order to communicate with the at least one medical device 20. Moreover, information may also be communicated in some exemplary embodiments with other components via the one interface 12 or the plurality of interfaces 12, e.g., for the subsequent further processing of the image data, for example, on a display or on a monitor, a display device, a memory device, an alarm generation device or a documentation system.

The interface 12 may correspond, for example, to one or more inputs and/or to one or more outputs for receiving and/or transmitting information, e.g., in digital bit values, analog signals, magnetic fields, based on a code, within a module, between modules, or between modules of different entities. However, the interface 12 may, however, also correspond to an input interface 12, such as a control panel, a switch or rotary switch, a knob, a touch-sensitive screen (also called touchscreen), etc. The interface 12 thus permits the recording, optionally also reception or input, sending or output of information, for example, for communication with the medical device 20. The interface 12 may have a wired or wireless configuration. This also applies to the interface 22 explained below at the medical device.

The computer 14 may be coupled to the interface 12 and to a detection device 16. In exemplary embodiments, the computer 14 may correspond to any desired controller or processor or a programmable hardware component. For example, the computer or determination device 14 may also be embodied as software, which is programmed for a corresponding hardware component. The computer 14 may be implemented to this extent as programmable hardware with correspondingly adapted software. Any desired processors, such as digital signal processors (DSPs) or graphics processors may be used here. Exemplary embodiments are not limited here to a certain type of processor. Any desired processors or even a plurality of processors may be employed for implementing the computer 14. FIG. 1 further shows that the computer 14 may be coupled to the detection device 16 in some exemplary embodiments. For example, one or more sensors of the detection device 16 detect at least three-dimensional (partial) image data in such an exemplary embodiment and provide these for the computer 14. The image data may comprise, for example, information about a patient positioning device, health care staff and medical devices.

FIG. 1 also shows a medical device 20 that is configured to receive information about a user via an interface 22 and a network, the information about the user indicating whether the user is located in an area surrounding the medical device 20. The medical device 20 is configured to use display information for a data output 24 based on the information about the user. The interface 22 may be implemented in the same or similar manner as the interface 12 explained above, for example, as a wired or wireless interface. Examples of such interfaces 12, 22 are Ethernet, Wireless LAN (Local Area Network), internet interfaces, mobile wireless device, etc. The medical device may likewise comprise a computer or processor 23 for data processing, corresponding to the above-described computer 14. The data output of the medical device 20 may be to a display that is a part of the device 20 or may be an external display, or to an external device or to other devices connected to the network via the interface 22.

The device 10 comprises in some exemplary embodiments a detection device 16 for detecting the optical image data of the medical device and of the area surrounding the medical device 20, wherein the detection device 16 has one or more sensors, which is/are configured to detect a three-dimensional point cloud as image data, as this is optionally shown in FIG. 1 (optional components are shown by broken lines in FIG. 1). The detection device 16 may correspond here to one or to a plurality of any desired optical detection units, detection devices, detection modules, sensors, etc. Cameras, image sensors, infrared sensors, sensors for detecting one-, two-, three- or more than three-dimensional data, sensor elements of various types, etc., are conceivable as well. The one or more sensors may comprise in other exemplary embodiments at least one sensor that supplies at least three-dimensional data. The three-dimensional data consequently detect information about pixels in the space and may comprise, quasi as additional dimensions, additional information, for example, color information (e.g., red, green, blue (RGB) color space), infrared intensity, transparency information (e.g., alpha values), etc.

There are various types of sensors, which, though not generating a two-dimensional image of a scene, do generate a three-dimensional set of points, e.g., pixels with coordinates or different depth information, which comprise information about surface points of an object. For example, information may be present here about a distance of the pixels from the sensor or sensor system. There are some sensors that record not only a two-dimensional image, but additionally a depth map, which contains the distances of the pixels from the sensor system itself. A three-dimensional point cloud, which represents the recorded scene in 3D (three-dimensionally), can then be calculated from this.

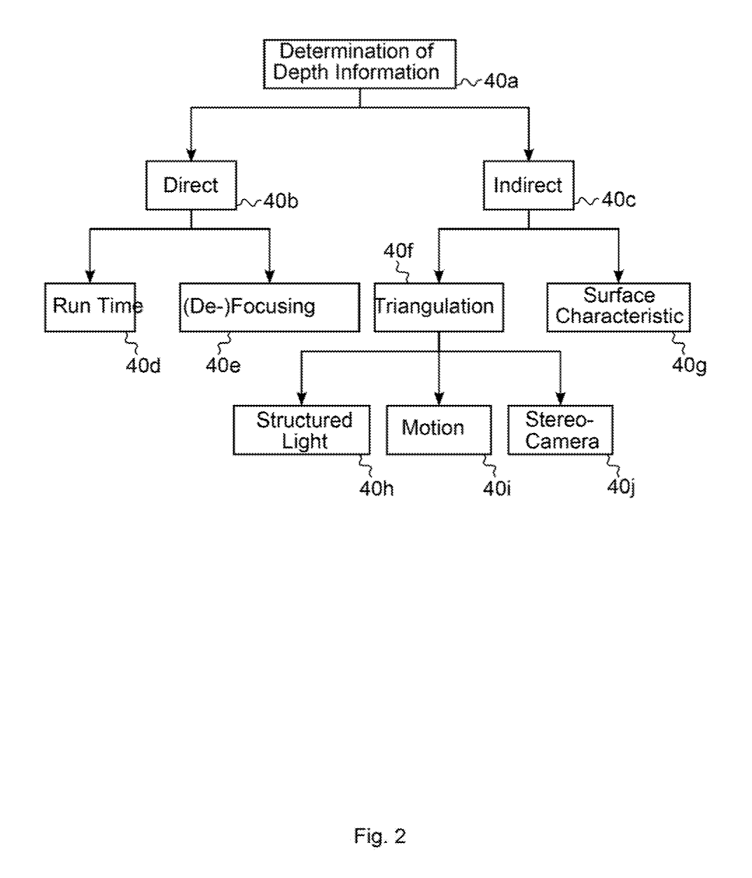

An overview of the different methods for determining the depth information for the depth map is shown in FIG. 2. FIG. 2 shows an overview diagram for determining three-dimensional image data in some exemplary embodiments. Distinction may be made between direct and indirect methods, where the distance of a point from the system is determined directly by the system itself in case of direct methods, and additional methods are needed in case of the indirect methods. Additional information about the individual possibilities can be found in, among others, in (Hartman, 2011). These sensors have become more favorable and better in the recent past. The three-dimensional information enables computers to analyze recorded objects more accurately and to derive information of interest, such as distances between objects.

FIG. 2 shows an overview diagram for determining three-dimensional image data in some exemplary embodiments, where determination variants going beyond FIG. 2 may also be used in exemplary embodiments. It should be noted that the three-dimensional image data, to which reference is being made here, often correspond only to a three-dimensional partial image, because a sensor determines pixels from a defined perspective only and an incomplete three-dimensional image may thus be formed. As will still be explained later, a plurality of such partial images may also be combined in order to obtain an image with improved quality or more pixels, which may, in turn, also correspond to a partial image only.

FIG. 2 first shows in 40a the determination or calculation of depth information in image data. Direct methods in branch 40b and indirect methods in branch 40c can be differentiated here, the former determining the distance of a point from the system directly via the system, and the latter requiring additional devices for the determination of the distance. Direct methods are, for example, time of flight methods 40d and (de)focusing methods 40e. Indirect methods comprise, for example, triangulation 40f (for example, by means of structured light 40h, motion 40i or stereo cameras 40j) and analysis of surface characteristics 40g. Further information about image data acquisition and image processing in this connection can be found in DE 10 2015 013 031.5 (corresponding U.S. application US2017103524 is hereby incorporated by reference in its entirety).

User interfaces are a means for making possible an interaction between a user and the devices and may occur in different types. Examples are a graphic user interface, touchscreen (touch-sensitive display or user interface), hardware interfaces, such as buttons, switches, rotary controls, slider controls, acoustic interfaces, etc. Adaptive user interfaces are a rather recent concept, in which the respective interfaces can be adapted to the needs of a user or to a context. The contents of a user interface can be adapted in case of adaptive presentation and the goal in adaptive navigation is to adapt a path to a target to be reached, e.g., Ramachandran, K. (2009), “Adaptive user interfaces for health care applications.”

A user is often identified and at least categorized by such devices in order to correspondingly adapt the user interface. This may make possible an adaptation to the level of experience of the user, filter mechanisms based on user preferences or recommendations (e.g., user-specific message filters), etc. Another aspect is security, because only functionalities for which the user in question is authorized can be offered to certain users.

Some related aspects can be found in U.S. Pat. No. 8,890,812 A1 “Graphical user interface adjusting to a change of user's disposition” (U.S. Pat. No. 8,890,812 is hereby incorporated by reference in its entirety) Sensors directly integrated in the medical devices are used here to adapt graphic user surfaces.

The sensors may be operated in exemplary embodiments uncoupled from the medical devices, so that a plurality of medical devices can be addressed by the same sensors or a plurality of sensors can also detect data for one medical device. The computer 14 may be configured to determine, based on the image data, that the user is located in the area surrounding a plurality of medical devices and to use another medical device (e.g., a monitor or a display) based on a detected interaction of the user with a medical device (e.g., an input device).

Exemplary embodiments provide a concept for the communication or configuration of medical devices, and content information and/or information about the surroundings (e.g., distance in space between device and health care staff, viewing direction, orientation of the body of the health care staff, etc.) of a medical device can be used to make possible, for example, an adaptation or a communication with the medical device or with the user interface thereof even under changing light conditions (daytime and nighttime). Exemplary embodiments consequently provide a system that may comprise uncoupled medical devices, sensor system and control/regulation. Exemplary embodiments may therefore be able to provide corresponding image data for one or more medical devices, which were potentially detected in the range of action of a nursing staff member. Such a potential identification can be made dependent on a distance between the nursing staff member and an identified medical device.

A method for configuring a medical device may comprise, for example, the following steps in exemplary embodiments:
1) Detection of depth information via sensors and potential combination of the detected image data into a point cloud;
2) Detection (segmentation and classification) of medical devices with user interfaces and the area surrounding them, as well as of persons outside a patient positioning device in the vicinity of or in the area surrounding the medical devices;
3) Linking of the medical device with network computers;
4) Determination of distances between the persons and the medical devices;
5) Determination of viewing directions or fields of view of the persons in the room, for example, by determining the position of the head, the orientation of the head, the orientation of the body, of the angle of view, etc.;
6) Storage of a time stamp;
7) Preprocessing of the data to obtain more information; and
8) Communication of the data from steps 4, 5, 6 and 7 to a network with the use of step 3 to bring the information to the correct recipients.

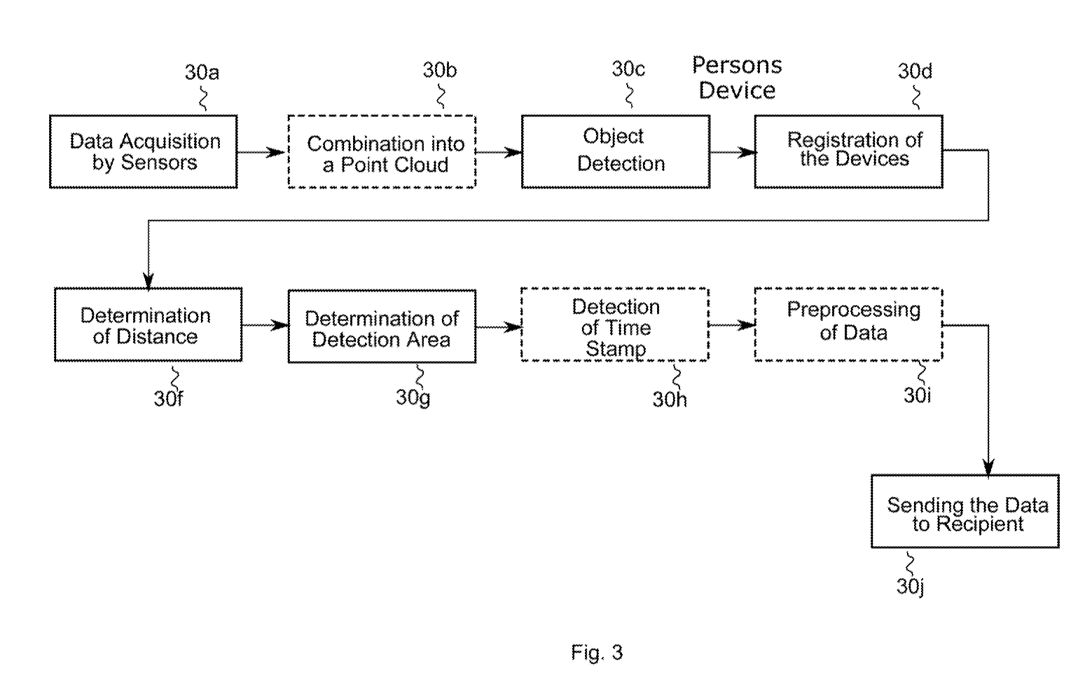

FIG. 3 shows a flow chart of a method in an exemplary embodiment. Data acquisition is first performed in a step 30a by sensors, for example, in the form of three-dimensional image data. These are then optionally combined into a three-dimensional point cloud 30b. Objects (e.g., medical devices, persons, health care staff members, patient positioning devices, etc.) can then be detected thereafter in step 30c. Information about the detected persons and devices can then be processed further later. A registration or pairing (also pairing) can then take place in a step 30d, in which the device 10 addresses the medical devices and a communication context is generated (in the sense of mutual disclosure). A distance determination 30f, e.g., between detected persons and medical devices, can subsequently take place, for example, with image processors. The computer 14 is now configured to determine a distance between the user and the at least one medical device 20 from the image data. Moreover, a detection field or detection area of the persons (e.g., viewing direction and angle of view, body or head orientation) can also be determined 30g. Likewise optionally, the information thus determined may optionally be provided with a time stamp. The data may then be processed further 30i before they are sent to the corresponding (legitimized or detected) recipients.

Such a method may be carried out in exemplary embodiments iteratively at different times. It is not necessary now to carry out all the above-described steps. A device may comprise, for example, the following components in exemplary embodiments. Reference is also made in this connection to the document DE 10 2015 013 031.5, which pertains to the determination of segment component positions of a patient positioning device based on image data.

In some exemplary embodiments, the device 10 may be coupled to 1 . . . n (n is a positive integer) sensors. The device 10 may comprise a detection device 16, which in turn comprises the sensors. The sensors detect a point cloud, which is based on combined image data of a plurality of sensors. Preprocessing of the image data can be carried out to this extent in the sense of the combination of the image data into a three-dimensional point cloud. The sensors may be arranged such that a room with the medical devices and with a patient positioning device are detected with a corresponding resolution.

Moreover, 1 . . . m (m being a positive integer) medical devices 20 may be present in exemplary embodiments in such a room or detection area to be monitored (optionally also a plurality of rooms). The medical devices 20 may comprise each one or more adaptable user interfaces. The medical devices 20 can then be addressed by the device, for example, via a network. The addressing information may comprise, for example, one or more elements of the group of information about a type of the medical device, information about a network address of the medical device, information about a specification of the medical device or information about the ability to reach or the configuration of the medical device.

The computer 14 may be implemented in such an exemplary embodiment comprising a processor, which is coupled to or connected to the 1 . . . n sensors (of the detection device 16). The method is implemented as software now and is carried out on the basis of the preprocessed point cloud of the image data of the sensors. The determined context information is passed on to the 1 . . . m devices 20 via a communication link (e.g., Ethernet), so that these are set up or configured based on this information (for example, their user interfaces, displays, modes, etc.).

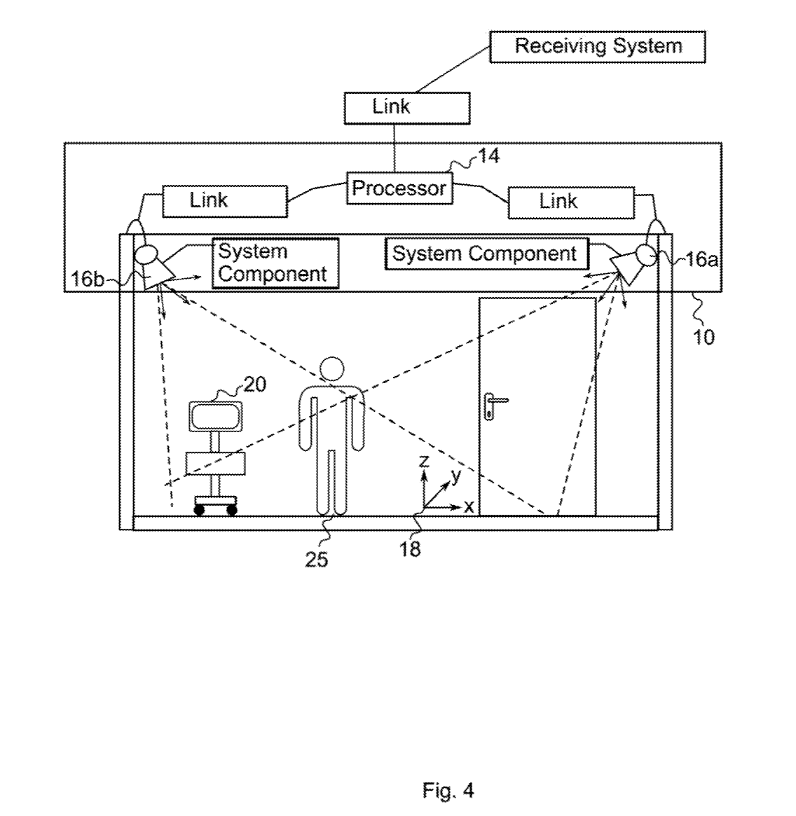

FIG. 4 shows an exemplary embodiment in a hospital room and represents a conceptual overview. FIG. 4 shows a hospital room with a medical device 20 and a detection device, which is implemented by means of two sensors of system components, for example, infrared sensors 16a and 16b. The device comprises two links (via interface 12) between a processor 14 (computer) and the sensors 16a, 16b. Moreover, there is a link between the processor 14 and the receiving system, which can, in turn, build up a link to the medical device 20 via an interface, not shown. The computer 14 is configured in some exemplary embodiments to build up a link to the at least one medical device 20 via the at least one interface 12 when a user is located in the area surrounding the at least one medical device 20. As is further shown in FIG. 4, a person 25, e.g., a nursing staff member, is also located in the hospital room. Moreover, one or more patients and patient positioning devices (hospital beds, operating tables, day-beds, etc.) may be located in the hospital room. The detected point cloud is based on the coordinate system 18.

In the exemplary embodiment shown in FIG. 4, the two sensors 16a and 16b detect a large part of the hospital room, which is being monitored here. The processor 14, which is coupled to the sensors 16a and 16b, then carries out one of the methods being explained here.

Exemplary embodiments can set or adapt user interfaces of medical devices by means of the context information provided. As a result, nuisance or disturbance caused to patients by the devices can be reduced; for example, displays can be switched off or faded out, noise levels can be reduced in the absence of the health care staff or adapted to the circumstances. The computer 14 is now configured to determine the absence of the user in the area surrounding the at least one medical device 20 and to change a setting of the at least one medical device 20 when the absence of the user was determined.

The legibility and interpretability of information relevant to the health care staff may possibly be improved in exemplary embodiments (e.g., by adapting font sizes of important information when a health care staff member is located farther away from a display). The device 10 may communicate with a plurality of medical devices, and a plurality of persons may also be detected with the detection device 16, so that the cost efficiency can be improved at least in some exemplary embodiments. The cleaning effort may be able to be reduced, because the device and the sensors or the detection device do not need to be arranged in the immediate vicinity of a patient. The medical devices themselves may become more favorable, because the need for internal sensors or cameras may be eliminated. Moreover, the maintenance effort (e.g., via software update) may be reduced in software implementations.

An exemplary embodiment of a method will be described below in detail. 1 . . . n sensors may be used to make it possible to acquire the image data. A piece of depth information can thus be detected per sensor, and a respective three-dimensional point cloud of the scene can be generated on the basis of this information. The point clouds thus detected can be combined into a point cloud based on a common coordinate system 18, cf. FIG. 4, into which the individual point clouds of the sensors can be transformed. For example, a stereo calibration may be performed, whereby a relative translation and rotation of a camera pair is determined. The point clouds can then be combined with means of linear algebra (translation and rotation matrices). Many sensors provide additional information. For example, infrared data can additionally be used and analyzed.

Different object detectors may be used in exemplary embodiments to detect persons and devices in the scene, in analogy to the data formats and conditions of the scene to be examined. The computer 14 is configured in these as to detect the at least one medical device 20 in the image data and to determine a position of the at least one medical device 20. The computer 14 can then be configured to set the at least one medical device 20 for operation by the user and/or for the output of information for the user when the user is located in the area surrounding a medical device 20.

For example, an iterative “closest point” algorithm (also “Iterative Closest Point (ICP)”), cf. Besl, Paul, may be used for static objects. A three-dimensional (3D) model of an object (e.g., a monitor) is searched for in the point cloud. If there is an agreement with a sufficient reliability, the object is detected. Another possibility is “keypoint” or 3D feature matching. 3D features are calculated here for known objects in the given point cloud, and a model can subsequently be scanned in the point cloud based on a point-to-point correspondence between the models and the point cloud. One example of such an algorithm is called “SHOT,” cf. Tombari et al. Additional 3D detectors and their matching can be found in Alexandre, Luis et al.

Another possibility is the use of the Hough transformation, cf. Woodford, Oliver et al., or implicit shape models, cf. Velizhev, Alexander et al., to assess and to detect objects in a scene.

Activities in the field of computer-based detection (also computer vision) were focused in the past on the area of object detection in two-dimensional (2D) image data. Some of these algorithms were adapted to the use of depth information and 3D image data. For example, S. Gupta et al. use a neural convolutional network to calculate features based on depth information and then to perform an object detection in the 2D area. A subsequent step marks pixels with a “decision forest” algorithm, which segments the objects. Gupta et al. develop this procedure further with a 3D model, where objects are then represented in the scene by model objects of a library in order to achieve an approximation to the actual conditions. Finally, it is also possible to perform directly a data-based classification in the 3D point cloud. S. Song et al. use an algorithm that is based on a shiftable window and they furnish hand-made features for the direct classification. S. Song develop this procedure further by combination with the above-mentioned algorithms. Some of these algorithms yield no 3D segmentation of the object, but they yield a 3D limitation frame for the objects within the point cloud. If a segmentation is necessary, for example, the segmentation step of S. Gupta et al. may be used.

The algorithms mentioned here may be used in exemplary embodiments to detect persons and devices. Based on the many poses and postures, which may occur in persons, especially the data-based methods (classification in 2D, transformation into 3D and direct classification in 3D) may be used for persons. It is possible to use, for example, the Kinect SDK (Software Development Kit) algorithm to make it possible to detect persons, their pose or posture, position, as well as individual body parts. The computer 14 is configured in some exemplary embodiments to determine a viewing direction and/or a body orientation of the user from the image data in order to infer the presence of an operating and/or reading intention of the user from the viewing direction, field of view and/or the body orientation, and to set the medical device 20 for the operating or reading intention when this is present.

The registration or pairing of devices with network computers may be carried out in an automated manner in exemplary embodiments. To make it possible to send the data, e.g., distances, viewing directions, etc., to the correct recipients, a pairing or association of the devices/objects within a network with visual objects (monitors/displays) is carried out in a virtual scene (computer-based representation of a real scene, for example, medical setting). This step is also called pairing. There are various possibilities for this step in exemplary embodiments as well.

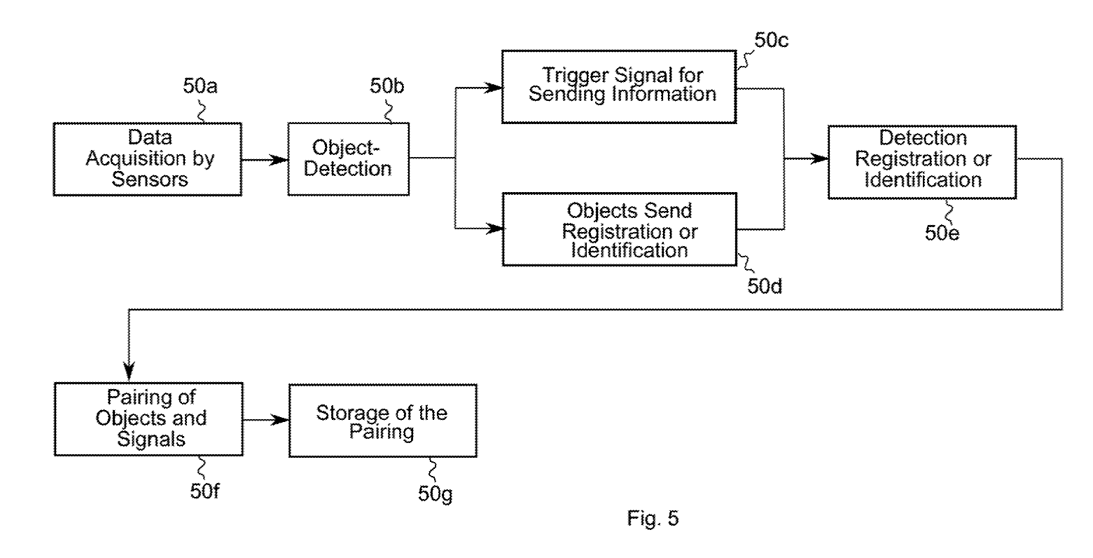

FIG. 5 shows a flow chart for the registration, detection or pairing of medical devices 20 via a network with the use of optical or acoustic identifications in an exemplary embodiment. The computer 14 is configured here to receive an identification from the medical device 20. The medical device 20 may be configured to send a registration signal for the medical device 20 for the medical device to register itself in the computer 14.

The method being considered begins with the data acquisition 50a by the sensors and is continued with the object detection 50b in the scene in question. Two possibilities are then taken into consideration. The device 10 sends a trigger signal from 50c, which triggers on the part of the medical device the sending of an identification (“pairing” signal, for example, optically or acoustically, light and/or audio signal). The identification may correspond to an optical signal, which is detectable in the image data, and/or to an acoustic signal. The medical device 20 may analogously be configured to receive a trigger signal via the network and to send an identification in response to the trigger signal. The medical device 20 may be configured to send an identification of the medical device 20 subsequent to the registration signal.

In addition or as an alternative, the objects or devices may also send a “pairing” signal (identification, registration signal) 50d without a dedicated trigger signal, for example, at regular intervals, based on a recurring control signal, when a new device is switched on or at the time of connection to a network. The “pairing” signal of the objects in the scene can then be detected by the device 10, 50e. The signals are subsequently paired or associated with the objects in the scene, 50f. Finally, the information about the linking/association/pairing may be stored, 50g, for example, for documentation purposes or for future processes. The device 10 may consequently further comprise a storage device, which is configured to store data, the computer 14 being designed to store information about the medical device by means of the storage device. The storage device is then coupled to the computer 14 and may be embodied as any desired storage device. Examples are hard drive storage devices, read-only memories, optical memories, etc.

For example, image data material, on the basis of which a virtual scene can be generated for the representation of the real scene, is detected in step 50a with 1 . . . n sensors. A plurality of sensor groups or sensor sets may also be used to generate a plurality of virtual representations for a plurality of real scenes (e.g., a plurality of rooms). The positions or locations of the objects in the virtual scene can be determined during the object detection 50b. The computer 14 is now configured to locate and/or identify the medical device 20 based on the identification and the image data. A request (trigger signal of the device 10) can be sent to the M detected objects via a network or a link in order to prompt these to send a known registration signal or an identification, 50c, for example, an unambiguous light or audio signal. The computer 14 is now configured to send a trigger signal for sending an identification via the at least one interface 12 to the at least one medical device 20.

As an alternative or also in addition, the M objects may also send the signals independently (a trigger would only be on the part of the objects) 50d. The objects could optionally also inform the device 10 via the network or the link that a “pairing” signal will be sent shortly. The “pairing” signal is then detected in the virtual scene, 50e. For example, an unambiguous signature may be used for this. In addition, a position or location of the respective object in the scene is determined. The identifications are then paired or associated with the objects in the virtual scene, 50f, based on the unambiguous signal and the position of the object in the scene. This information may then be stored for later use. The computer 14 is now configured to receive an identification of the at least one medical device 20 via the at least one interface 12, which indicates that the at least one medical device 20 would like to register itself. The computer 14 is now configured to receive an identification of the at least one medical device 20 subsequent to the registration signal.

For example, the camera-based system (device 10 with camera-based detection device 16) may perform a “pairing” for 1 . . . M detected objects in the scene. It may be known, for example, that a visual object without association is a ventilator and a trigger signal can then be set to all ventilators in the network in order for an identification to be subsequently detected by this non-associated object as well. The device may send a trigger signal to all objects in a room (or to all objects for which the room is unknown) in another exemplary embodiment in order for the identifications to be subsequently detected by all these objects. Known objects may optionally be pulled off/removed in advance and the sending of an identification may then be requested by the other objects.

To accomplish this, a network protocol may make provisions for addressing objects of a certain type T or in a certain room. Corresponding identifications or signatures can then be determined for these objects and the sending of the identifications can be requested at the objects. These steps may be performed by protocols with discovery functionality (also called discovery), one example being DPWS (Devices Profile for Web Service). The signature will not necessarily be explicitly signalized in advance in some exemplary embodiments, and it may also arise from a network address or the addressing information or it may be able to be determined on the basis thereof. It was considered so far that the camera-based system triggers the communication with the objects. The trigger may also take place in some exemplary embodiments on the part of the objects. An object or device, which has just come into the network, can then send a type of “hello” message with the subsequent sending of the signature or identification (light/audio). The camera-based system can receive the “hello” message and subsequently attempt to detect the identification.

If an object or device sends the identification, the camera-based system attempts to detect this in the scene. Sensors may be used now to detect the image and sound. The sensors may also have infrared detection, i.e., detection of infrared light (IR) with a wavelength of, for example, 827-850 nm (invisible range). The devices or objects may be equipped with corresponding infrared emitters (IR emitter, IR diode, etc.) in order to be able to send IR signal. The image data may consequently also comprise infrared image data.

By switching the IR source on and off, a device can then send an identification or signature of a certain duration or with a repetition rate that is detected on the part of the sensor and then detected. The sensor signals now reflect the location and the particular state of the IR source. Image-processors may then be used for the further processing and for the detection of the signature (light source, bright points in the scene). This may also be carried out repeatedly, in which case the results may be combined in order to detect the signatures.

In case audio signals are used, the devices or objects are equipped, for example, with loudspeakers. The audio signals are likewise detected now, for example, by the camera system with integrated microphones. The signals may be outside an audible range (approx. 20 Hz to 20 kHz). The signature may now consist of a sequence of different audio frequencies or of a certain frequency (frequency combination), which is sent over a certain time. Combinations are likewise conceivable. The at least one interface 12 may now be configured, furthermore, to receive audio data, and the computer 14 may be configured to analyze the audio data with respect to an audio identification of the at least one medical device 20.

The associations can be generated with the detected locations and objects and the addressing information for the objects. This may be achieved by signal localization and position comparison. For example, all the objects that may be considered as a signal source may be determined for each signal, e.g., by determining the respective limitation frames in the point cloud. If only one visual object is present, the association is obvious, If a plurality of objects with overlapping limitation frames are present, for example, the object with the smaller limitation frame may be used or a fine localization (a plurality of localizations and combination of the results) may be performed. A 1-to-1 imaging is thus achieved between the identifications and the objects in some exemplary embodiments. This information may then be stored, for example, in the form of a table (limitation frame, set of points for the object, network address, etc.). The camera-based system (device 10) will have access to this information from then on.

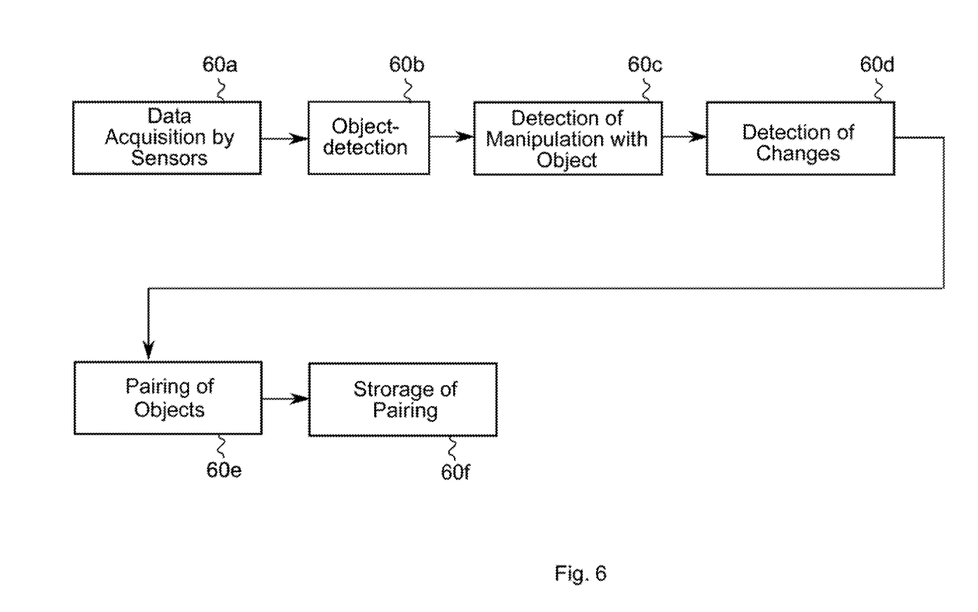

FIG. 6 shows a flow chart for registering, detecting or pairing medical devices 20 via a network with the detection of manipulations with the objects in an exemplary embodiment. The data are first detected in a step 60a via the sensors and an object detection 60b is performed. A manipulation with the visual objects is then detected, 60c in the image data. The changes in the arrangement or configuration of the objects, 60d, are detected, and the effects on the pairings, 60e, are then determined. The changed pairing information is then updated.

Image data are again detected in step 60a by means of the 1 . . . n sensors and a virtual scene is generated. A plurality of sensor groups may again be used to this end, for example, for a plurality of rooms. The objects in the virtual scene are detected, 60b, and any manipulations are determined, 60c. It is determined whether a manual manipulation is present and to what extent this becomes visible through the object in the virtual scene. The visual objects may then be paired with the network entities, 60c, and the information may be stored, 60f. The detection of the real scene and the detection of the objects in the scene may be carried out as described above. The camera-based system now determines whether a visual object was manipulated in the virtual representation of the real scene. This may be carried out by means of various visual notes in the image data. For example,
1) should a person who is not a patient be located in the vicinity of the object/device,
2) this should also happen over a certain time period (e.g., t seconds),
3) the person should look at the device, i.e., the view of the person should be directed towards the device,
4) the person should touch the device, and
5) then the display of the device should change.

The more these conditions apply, the more reliable is the detection of an object manipulation. The first condition can be detected in a simple manner after the objects in the scene were detected. Distances in the virtual representation, which were detected with a calibrated 3D sensor, correspond essentially to the real distances. Further details concerning the determination of the distances between persons will be explained below. The second condition can be checked by repeatedly checking the first condition over a time period. Details concerning the detection of the field of view will likewise be explained later. Whether there is a contact between a person and a device can be found out by repeatedly determining the distance and by comparing the distance with a threshold value. The computer 14 may now be configured to determine a contact/interaction between the at least one medical device 20 and the user on the basis of the image data and to communicate with the at least one medical device 20 based on a determined contact and/or interaction by the user.

This may, however, be difficult if there is no direct visual contact between the sensors and the location of the contact/interaction, and this condition may not be absolutely necessary in some exemplary embodiments. A change in a display at a medical device may be interpreted as an indication of a manipulation. Detection of a display unit or display may be carried out similarly to the detection of a person, cf. Shotton J. There likewise are several possibilities for quantifying such a change, and “vibeinmotion” would be an example. Whether changes on a monitor are, indeed, visible in the image data also depends on the luminosity of the monitor and the sensitivity of the camera or of the sensor.

A network device can, moreover, disclose changes in the network, for example, by sending an indicator that indicates that a manual manipulation has taken place. The camera-based system can then receive such an indicator and store the change together with a time stamp and the network address of the device. The computer 14 is configured in this case to store a time stamp with the information about the medical device 20. Moreover, an association may be established between a network object A and a visual object V based on the manipulation and based on whether this took place in the same time period in A and V. If additional indications are available, room and type of an object, these can also be used to resolve ambiguities. If an association is made, this is unambiguous in at least some exemplary embodiments. This may then be stored in a table, which can be read at least by the camera system.

After the objects, which represent the m devices and n persons in the point cloud, have been found, their distances can be calculated. The distances of the 3D points agree with the real distances within the range of the accuracy of the calibration and the resolution of the sensors. One possibility to determine the distance is to determine the centers of the gravity of the objects. These can be determined/estimated by determining the centers of gravity of the limitation frames or the centers of gravity of a set of points that represents the object. Then, n centers of gravity for the persons and m centers of gravity for the devices and the respective distances can be determined from this.

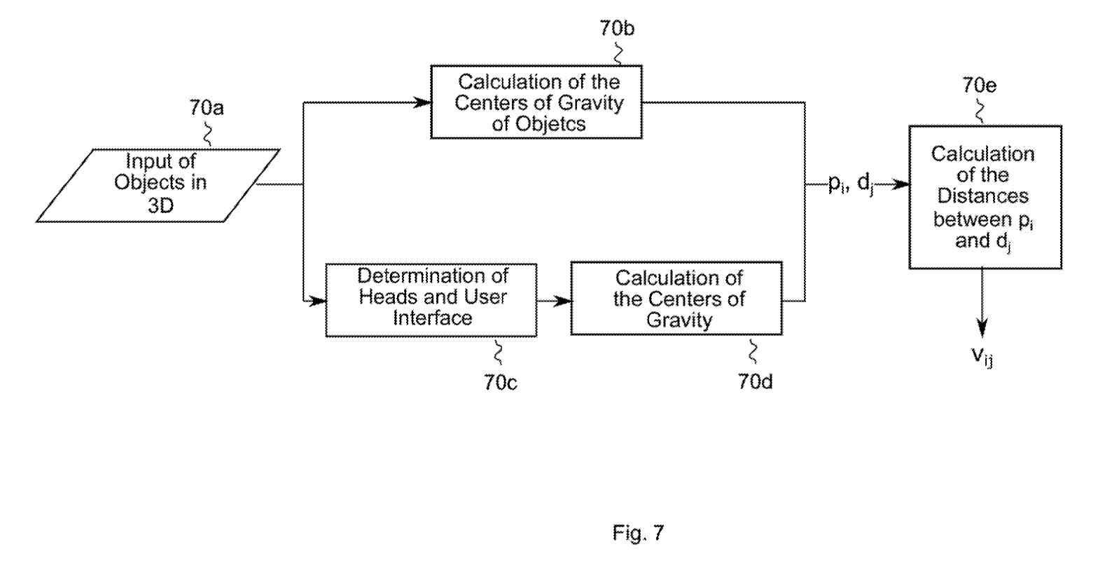

FIG. 7 shows a flow chart for determining distances between a health care staff member and a medical device 20. The method begins with the input of the 3D objects, 70a. The centers of gravity of the objects are then calculated, 70b, in one variant. As an alternative or in addition, it is also possible to determine the heads of the persons and the user interfaces, 70c. The centers of gravity of the head and user interfaces can be determined after this, 70d. Based on the centers of gravity pi and dⱼ thus obtained, the distances vᵢⱼ between pᵢ and dⱼ can be determined, 70e. The positions or locations of the heads of the persons and of the user interfaces are then determined in one exemplary embodiment. Based on this, the centers of gravity of the point clouds of the objects, which result from this, and the resulting distances can be determined (lower path in FIG. 7). It is possible to use, for example, known face detection mechanisms to make it possible to identify the heads. Examples can be found in Viola P. et al. In addition or as an alternative, a segment detection of previously detected persons and body part detection may be carried out as well, cf. Shotton et al., which is a part of Kinect SDK.

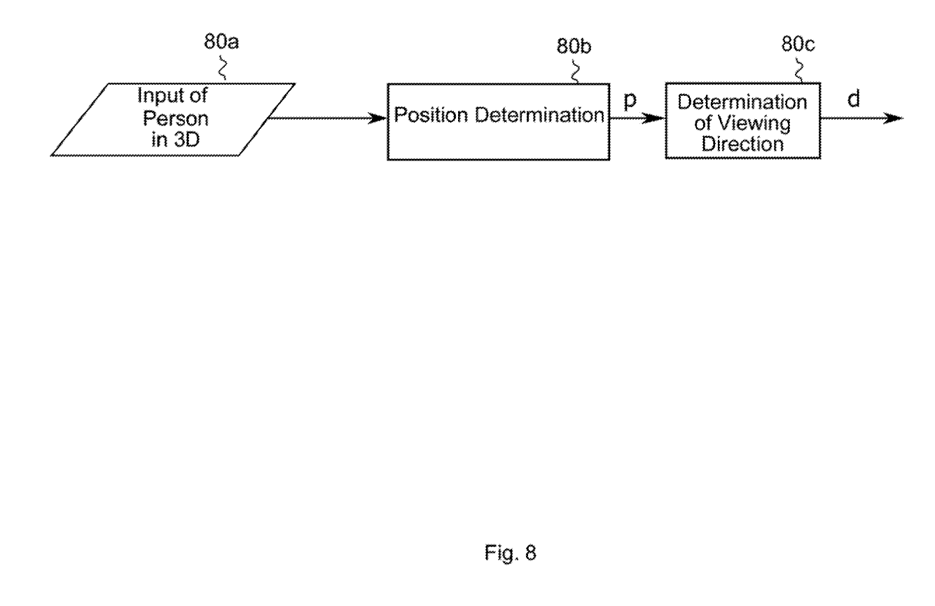

The field of view of the person is additionally analyzed in some exemplary embodiments, for example, by detection of the head or body position, determination of the angle of view, etc. The fields of view of persons in the room depend at least on the positions of the persons and their viewing direction, both of which can be taken into consideration in the determination of the field of view, as it is further illustrated in FIG. 8.

FIG. 8 shows a flow chart for determining a viewing direction or a field of view in one exemplary embodiment. The method shown begins with the input of the image data of the person in 3D, 80a. This is followed by the determination of the position, 80b, which yields the position p. A viewing direction d is determined thereafter, 80c. The output of the step before step 70c from FIG. 7, i.e., pᵢ, which are also designated by p for simplicity's sake, may be used to determine the position. The center of gravity of a person is thus assumed to be the center of gravity of his head. The viewing direction may be derived in different manners, depending on the desired precision and the desired effort. A first possibility would be to use a body orientation or head orientation of the person, which can be derived from a motion direction of the person (which presupposes monitoring of the motion of the person, for example, with Kinect SDK). Another, possibly better possibility is the estimation of the orientation of the head. Fanelli G. et al. determine depth information and “Random Regression Forests” to estimate the orientation of the head. The depth information may be obtained, for example, directly from the n sensors, as described above, or calculated from the individual point clouds of the sensors. The determination of the viewing direction can be further improved by the application of eye tracking (also eye tracker). Once the (estimated) head position and the (estimated) viewing direction have been determined, the field of view over the average human perception angle (eye aperture) can likewise be determined in the horizontal and vertical directions.

A time stamp may be detected and/or stored in exemplary embodiments. The device may comprise to this end a timer or a clock, which is coupled to the computer 14. This clock or the timer may be comprised in the computer 14, or it may also be available via the network.

The computer 14 may, moreover, also carry out one or more preprocessing steps in order to upgrade the data. Some or all of these preprocessing steps may also be carried out at another location, for example, at another computer unit in the network, by the detection device 16, or within the medical device. Possible preprocessing steps would be, e.g., the determination of whether a device 20 had already been in the field of view of a person before and when this happened (e.g., field of view, position of the device and time stamp). Another step would be the determination of a vector that describes the change of a field of view (velocity and motion direction), as will be described in more detail later.

Finally, a data transmission to the devices 30 or communication with the devices 20 is carried out in some exemplary embodiments. The determined or resulting data are transmitted to the corresponding devices 20. Since it is known from the “pairing” step which object/device belongs together with which network computer, reference may be made to the general network transmission protocols.

The described device 10 and the described methods determine, in at least some exemplary embodiments, the distances between m devices and n persons and provide, moreover, information about the corresponding network computers. D is one of the m devices, which receives a tuple (d₁, . . . , dₙ) of distances for n persons and comprises as information, for example,
1) a tuple of tuples that describe the fields of view (f₁, . . . , fₙ) of the persons, wherein fi indicates the respective head positions or head locations of the persons, the viewing direction and possibly a vertical and horizontal aperture angle and D's position p (unless known anyway), and/or
2) a tuple (b₁, . . . , bₙ), wherein bi indicates whether the device D is in the field of view of the person I.

The device 10 or a corresponding method can determine this information for a plurality of persons and a plurality of devices at the same time, and the devices do not have to be equipped with cameras or sensors of their own. For example, the device D calculates the (b₁, . . . , bₙ) itself based on the received (f₁, . . . , fₙ) and p. D can then determine a minimum distance from di, for which bi shows that D is in the field of view of the person p, and the minimum distance is also called M.

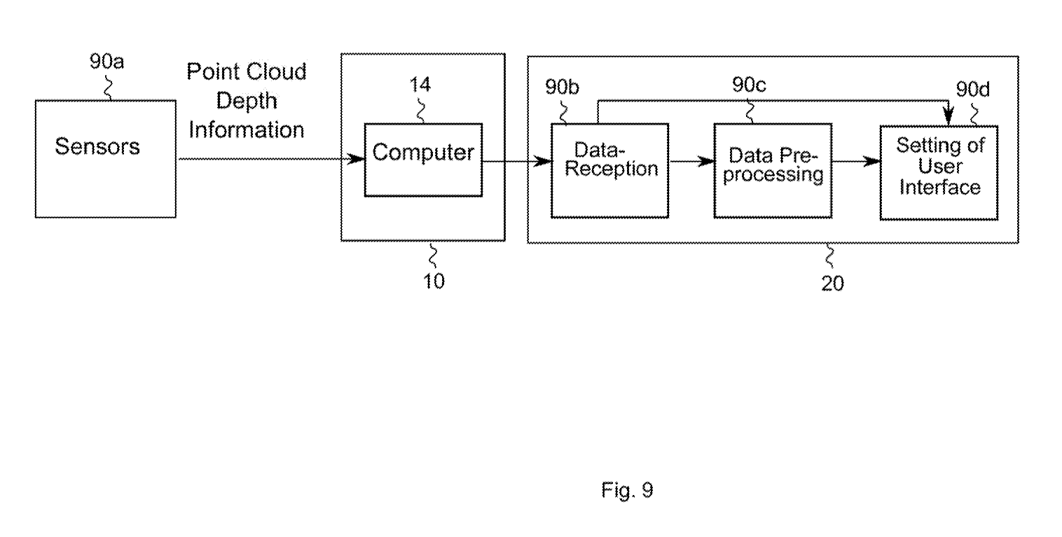

FIG. 9 shows a flow chart for adapting a user interface in an exemplary embodiment. Depending on the concrete information, which is present at D, preprocessing may also be unnecessary. FIG. 9 shows that sensors first detect a point cloud or depth information, 90a, and provide it for the computer 14 in the device 10. The medical device 20 receives the data preprocessed by the computer 14, 90b, it possibly performs a data preprocessing, and it finally sets the user interface, 90d. Some examples of user interface settings will be explained below in exemplary embodiments.
a) Using M, D sets its user interface as follows:
D is assumed to be a patient monitor, which can display a maximum of three parameters with corresponding curves, e.g., heart rate (pulse), respiration rate and oxygen saturation. If M becomes lower than a threshold value, D displays all three parameters along with the curves. Otherwise, D displays only the most important parameter, e.g., the heart rate, with a symbol size that increases linearly with M until a maximum symbol size is reached.
b) D could indicate a pseudo-3D display by the user interface elements being adapted based on the angle of view of the user. For example, C could represent a 3D model of an object, which is adapted based on the viewing direction, or D could carry out the view synthesis. This could happen, for example, by storing a group of images of the object from different perspectives, on the basis of which an artificial 3D image is determined, cf. Seitz et al.
c) D could also represent approximately the same view of an object, regardless of where the user is located. A 3D model could be used for this, whose image data are rotated or pivoted prior to the presentation on a display such that the same side of the object always points in the direction of the user.

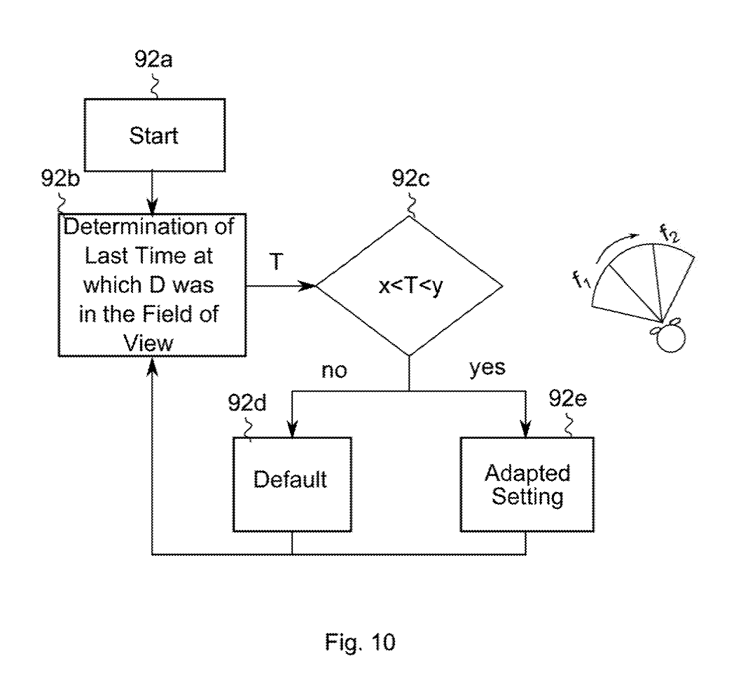

FIG. 10 shows a flow chart for avoiding too frequent adaptations of a user interface. FIG. 10 shows the flow chart on the left-hand side and on the right-hand side it shows how a prediction can be made in an exemplary embodiment, on the basis of a change in the angle of view, as to when a device is located in the field of view. The method makes provisions (FIG. 10, left), after a starting step 92a, for determining or calculating the last time at which D was in the field of view. The above-described situation is used as a starting point, in which the data contain a time stamp T. The computer 14 or a computer in the medical device 20 can then execute the following steps.
a) Determination of the last time at which D was in the field of view of the person (T seconds). In this exemplary embodiment, D would not set, for example, a default (also “default screen”), but it would remain at a setting 92e adapted to the user if the time period T is between the limits x and y, x<T<y, 92c. The intuition would be that D does not change its user interface too often if it is probable that it returns again into the field of view. A typical scenario would be a health care staff member who checks some devices at short intervals.
b) Determination of the changing viewing direction vectors. Taking a current field of view of the person and change vectors into consideration, D can make a prediction of when it returns into the field of view of the person and adapt the user interface correspondingly in advance. FIG. 10 shows on the right-hand side a person whose field of view vector changes from f₁ to f₂. This would likewise avoid too frequent changes in the user interface if the device was already in the field of view of the person. Exemplary embodiments can thus permit an adaptation of the user interface even if the person is not located in the detection range of a camera present in the device.

Deactivation of user interfaces may also take place in some exemplary embodiments. If the above situation is assumed, it can further be assumed that the device 10 notices that no person is located in the vicinity of or in the area surrounding the device, and the tuple with the distances would be empty in this case. The device D could then behave corresponding to one or more of the following possibilities.
a) If D is a display or a display unit, this can be switched off, so that patients are not disturbed by the emitted light or even to save energy.
b) If D reproduces sound signals or noises, the intensity of these signals or noises could be influenced, e.g., turned down in order not to disturb the patient, or turned up to make warning signals more perceptible for the health care staff outside the room as well.
c) If D has a keyboard, this can be switched off in order to prevent manipulations, for example, by patients.
d) If D has other interfaces, such as buttons, controls or switches, these may likewise be deactivated in order to prevent manipulations.

These actions can be canceled in exemplary embodiments when a health care staff member enters the room.

In medicine and pharmacology, titration is a method for adapting drug doses or parameter doses, and it is also referred to as dose titration. The dose of a parameter is regulated until an optimal or good result is obtained. A health care staff member will generally adapt to this end the dose of the parameter on a device (B) and check the effect of the adaptation on another device (A) at the same time. The device 10 and the methods being described here can support a health care staff member by the user interface of the device (A) being adapted when the health care staff member interacts with the device (B), so that the user interface of the device (A) reflects, for example, the effect of a changed setting on the device (B).

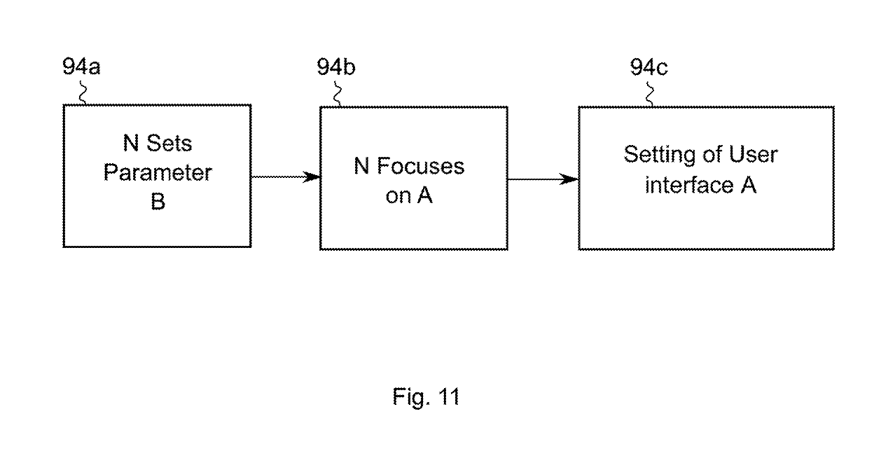

FIG. 11 shows a flow chart for adapting a user interface of a medical device A based on a parameter change at a medical device B in an exemplary embodiment. In a step 94a, a health care staff member N sets a parameter, e.g., FiO₂ (inspiratory oxygen level/oxygen fraction of a ventilated patient) on the device B, for example, on a ventilator. N then concentrates on the device A, 94b, for example, a patient monitor. The device 10 or a corresponding method then changes the setting of the user interface on the device A, 94c. The display on the device A is changed such that the respiratory physiological parameters of the patient, for example, an oxygen saturation, will predominantly be displayed. It would, moreover, also be conceivable to display the FiO₂ value itself on the patient monitor, so that N can observe the current value and its effect at the same time. Another variant would be a change from a time curve to a trend display. Another example would be an adaptation of a drug administration via an injection pump/syringe pump. If, for example, the device 10 detects a manipulation with the injection pump/syringe pump by a health care staff member in a syringe pump holder and it is known which syringe pumps are located in the holder (e.g., from the “pairing” step), the parameter now being changed can be determined and the now relevant physiological parameters can be predominantly displayed on a patient monitor. Such a detection is often impossible with cameras integrated in the medical devices.

Some medical devices are calibrated before they function in a reliable manner. Some devices can calibrate themselves, or a user sets a parameter on a first device a and observes the effect of the setting on a second device B. Exemplary embodiments may be useful in this procedure in the above-described manner, because the status of a calibration operation can be visualized in an automated manner. One example of such an operation is the zeroing of a patient monitor. A user initializes the zeroing, for example, by actuating a button intended for that purpose on the monitor. The user then changes the setting of a pressure transducer, for example, by opening a three-way valve to the ambient atmosphere, and then checks the calibration on the monitor. The system should not be changed during the calibration, e.g., the level at which the pressure transducer is located and the position of the patient should not be changed. More information can be found on this process in “Nursing Knowledge in Intermediate Care: For continued Education and Practice”, 2^{nd} edition, Chapter 2.4.2 Invasive Blood Pressure Measurement. Exemplary embodiments may be useful in this process as well, because the state of calibration can be visualized and manipulations with the pressure transducer or the patient during the process can be detected. Exemplary embodiments can then transmit warning signals or warning messages to the patient monitor, e.g., “Calibration invalid or failed, pressure transducer was moved.”

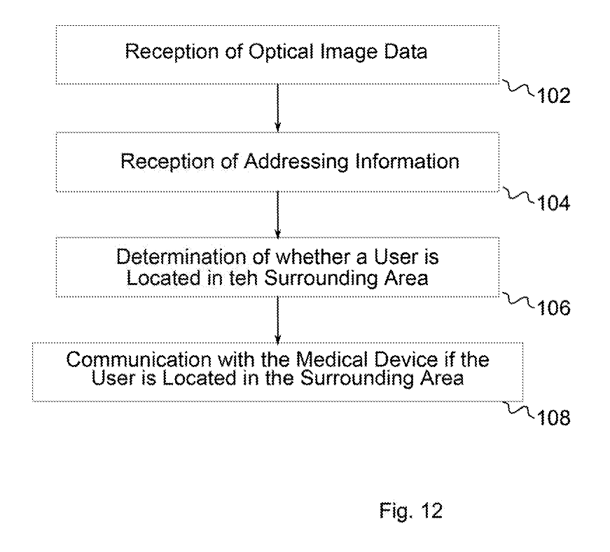

FIG. 12 shows a block diagram of a flow chart of an exemplary embodiment of a method for configuring a medical device 20. The method for configuring the at least one medical device 20 comprises the reception 102 of optical image data of the medical device 20 and of an area surrounding the medical device 20. The method further comprises the reception 104 of addressing information about the at least one medical device 20. The method further comprises the determination 106 of whether a user of the medical device 20 is located in the area surrounding the medical device 20 and a communication 108 with the at least one medical device 20 when the user is located in the area surrounding the medical device 20.

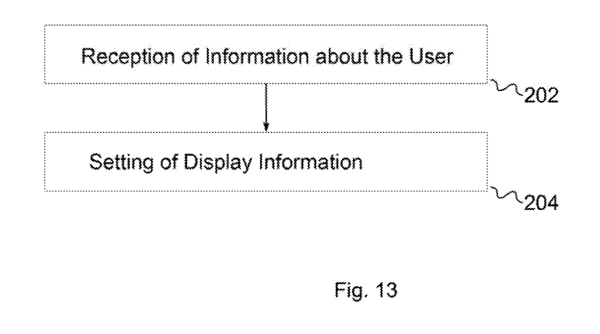

FIG. 13 shows a block diagram of a flow chart of an exemplary embodiment of a method for a medical device 20. The method for the medical device 20 comprises the reception 202 of information about a user, e.g., via a network and/or an interface 22, wherein the information about the user indicates whether the user is located in an area surrounding the medical device 20. The method further comprises a setting 204 of display information for a data output based on the information about the user.

Another exemplary embodiment is a program or computer program with a program code for executing one of the above-described methods when the program code is executed on a computer, on a processor or on a programmable hardware component.

The features disclosed in the above description, the claims and the drawings may be significant for the embodiment of exemplary embodiments in their different configurations both individually and in any desired combination and, unless states otherwise in the description, they may be combined with one another as desired.

Even though some aspects were described in connection with a device, it is obvious that these aspects also represent a description of the corresponding method, so that a block or a component of a device may also be defined as a corresponding method step or as a feature of a method step. Analogously to this, aspects that were described in connection with or as a method step also represent a description of a corresponding block or detail or feature of a corresponding device.

Depending on certain implementation requirements, exemplary embodiments of the present invention may be implemented in hardware or in software. The implementation may be carried out with the use of a digital storage medium, for example, a floppy disk, a DVD, a Blu-Ray disk, a CD, a ROM, a PROM, an EPROM, an EEPROM or a FLASH memory, a hard drive or another magnetic or optical storage device, on which electronically readable control signals are stored, which can or do interact with a programmable hardware component such that the method in question is executed.

A programmable hardware component may be formed by a processor, a computer processor (CPU=Central Processing Unit), a graphics processor (GPU=Graphics Processing Unit), a computer, a computer system, an application-specific circuit (ASIC=Application-Specific Integrated Circuit), an integrated circuit (IC=Integrated Circuit), a single-chip system (SOC=System on Chip), a programmable logic element or a field-programmable gate array with a microprocessor (FPGA=Field Programmable Gate Array).

The digital storage medium may therefore be machine- or computer-readable. Some exemplary embodiments consequently comprise a data storage medium, which has electronically readable control signals, which are capable of interacting with a programmable computer system or with a programmable hardware component such that one of the methods being described here is carried out. An exemplary embodiment is thus a data storage medium (or a digital storage medium or a computer-readable medium), on which the program for executing a method being described here is recorded.

Exemplary embodiments of the present invention may generally be implemented as program, firmware, computer program or computer program product with a program code or as data, wherein the program code or the data act such as to execute one of the methods when the program is run on a processor or on a programmable hardware component. The program code or the data may also be stored, for example, on a machine-readable storage medium or data storage medium. The program code or the data may be, among other things, as source code, machine code or byte code as well as other intermediate code.

Another exemplary embodiment is, furthermore, a data stream, a signal sequence or a sequence of signals, which stream or sequence represents one of the methods being described here. The data stream, the signal sequence or the sequence of signals may be configured, for example, such as to be transferred via a data communication link, for example, via, the internet or another network. Exemplary embodiments are thus also signal sequences, which represent data and are suitable for transmission via a network or a data communication link, wherein the data represent the program.

A program according to one exemplary embodiment may implement one of the methods during its execution, for example, by reading storage locations or writing a datum or a plurality of data into these, as a result of which switching operations or other procedures are brought about in transistor structures, in amplifier structures or in other electrical, optical, magnetic components or components according to another principle of operation. Data, values, sensor values or other information may correspondingly be detected, determined or measured by a program by reading a storage location. A program may therefore detect, determine or measure variables, values, measured variables and other information by reading one or more storage locations as well as bring about, prompt or execute an action by writing to one or more storage locations as well as actuate other devices, machines and components.

The above-described exemplary embodiments represent only an illustration of the principles of the present invention. It is obvious that modifications and variations of the arrangements and details being described here will be seen by other persons skilled in the art. The present invention is therefore intended to be limited only by the scope of protection of the following patent claims rather than by the specific details that were presented here on the basis of the description and the explanation of the exemplary embodiments.

While specific embodiments of the invention have been shown and described in detail to illustrate the application of the principles of the invention, it will be understood that the invention may be embodied otherwise without departing from such principles.

What is claimed is:

1. A device for configuring at least one medical device, the device for configuring comprising:
at least one interface for communication with the at least one medical device and for receiving optical image data of the at least one medical device and receiving optical image data of an area surrounding the medical device; and
a computer configured to control the at least one interface, to detect the at least one medical device in the image data, to determine a position of the at least one medical device, to determine whether a user of the medical device is located in an area surrounding the medical device, to determine a viewing direction of the user or a body orientation of the user or both a viewing direction of the user and a body orientation of the user from the image data in order to infer a presence of an operating intention of the user or reading intention of the user or both an operating intention of the user and a reading intention of the user from the viewing direction or from the body orientation, and to set the medical device based thereon, to communicate with the medical device when it is determined the user is located in the area surrounding the medical device and to receive addressing information about the at least one medical device via the at least one interface.

2. A device for configuring in accordance with claim 1, wherein the addressing information comprises one or more elements of a group of information about a type of the medical device, information about a network address of the medical device, information about a specification of the medical device or information about the ability to reach or configuration of the medical device.

3. A device for configuring in accordance with claim 1, further comprising a detection device configured to detect the optical image data of the medical device and to detect the optical image data of the area surrounding the medical device, wherein the detection device comprises one or more sensors configured to detect a three-dimensional point cloud as image data.

4. A device for configuring in accordance with claim 1, wherein the computer is configured to determine a distance between the user and the at least one medical device from the image data.

5. A device for configuring in accordance with claim 1, wherein the computer is configured to determine an absence of the user in the area surrounding the least one medical device and to change a setting of the at least one medical device based thereon.

6. A device for configuring in accordance with claim 1, wherein the computer is configured to receive an identification from the medical device.

7. A device for configuring in accordance with claim 6, wherein the computer is configured to locate the medical device or to identify the medical device or to both to locate the medical device and to identify the medical device based on the identification and the image data.

8. A device for configuring in accordance with claim 1, wherein the computer is configured to determine an interaction of the user with the at least one medical device and to communicate with the at least one medical device based on a determined interaction of the user.

9. A device for configuring in accordance with claim 1, wherein the at least one medical device is one of a plurality of medical devices and the computer is configured to determine on the basis of the image data that the user is located in the area surrounding a plurality of medical devices and to set another medical device based on a detected interaction of the user with the at least one medical device.

10. A device for configuring in accordance with claim 1, the computer is configured to set a plurality of medical devices for one or more users.

11. A medical device comprising:
an interface or a network connection configured to receive information about a user via an interface or a network or both an interface and a network, wherein the information about the user indicates whether the user is located in an area surrounding the medical device and indicates a viewing direction of the user or a body orientation of the user or both a viewing direction of the user and a body orientation of the user to infer a presence of an operating intention of the user or reading intention of the user or both an operating intention of the user and a reading intention of the user from the viewing direction or from the body orientation;
a medical device processor configured to set display information for a data output based on the information about the user.

12. A method for configuring a medical device, the method comprising:
receiving optical image data of the medical device and of an area surrounding the medical device;
receiving addressing information about the at least one medical device;
determining whether a user of the medical device is located in an area surrounding the medical device; and
communicating with the at least one medical device when the user is located in the area surrounding the medical device.

13. A method according to claim 12, wherein a program with program code executes at least a portion of the steps of receiving optical image data, receiving addressing information, determining whether a user of the medical device is located in an area surrounding the medical device and communicating and the program code is executed on a computer, a processor or a programmable hardware component or any combination thereof.

14. A medical device method comprising:
receiving information about a user, wherein the information about the user indicates whether the user is located in an area surrounding the medical device; and
setting of display information for a data output based on the information about the user.

15. A medical device method according to claim 14, wherein a program with program code executes at least a portion of the steps of receiving information about a user and setting of display information and the program code is executed on a computer, a processor or a programmable hardware component or any combination thereof.
